(19) United States
(12) Patent Application Publication
(10) Pub. No.: US 2022/0254508 A1
(43) Pub. Date: Aug. 11, 2022

(54) METHOD FOR IDENTIFYING MANIFOLD CLUSTERS USING STATISTICALLY SIGNIFICANT ASSOCIATION PATTERNS

(71) Applicant: SIPPA Solutions, LLC, Bayside, NY (US)

(72) Inventors: Bon K. Sy, Bayside, NY (US); Jin Chen, Bayside, NY (US)

(21) Appl. No.: 17/720,537

(22) Filed: Apr. 14, 2022

Related U.S. Application Data
(63) Continuation-in-part of application No. PCT/US2020/055616, filed on Oct. 14, 2020.
(60) Provisional application No. 62/914,594, filed on Oct. 14, 2019.

(51) Int. Cl. G16H 50/70

(52) U.S. Cl. CPC G16H 50/70

(57) ABSTRACT

A method for identifying a manifold cluster using statistically significant association patterns. A data set of real, continuous numbers is received and converted to corresponding discrete data representations. Statistically significant association patterns of the data are utilized to generate a manifold cluster. Customized actions, such as customized messages, are generated that are specific to the manifold cluster.

### CROSS-REFERENCE TO RELATED APPLICATIONS

This application claims priority to and is a continuation-in-part of international patent application PCT/US2020/055616 (filed Oct. 14, 2020) which claims priority to U.S. Patent Application 62/914,594 (filed Oct. 14, 2019), the entirety of which are incorporated herein by reference.

### STATEMENT OF FEDERALLY SPONSORED RESEARCH OR DEVELOPMENT

This invention was made with Government support under grant numbers 1648780 and 1831214 awarded by the National Science Foundation. The government has certain rights in the invention.

### BACKGROUND OF THE INVENTION

The total cost of health care services reported by the Center for Disease Control (CDC) in 2012 was $2.7 trillion. Of these expenditures, 86% were attributed to patients with chronic disease. Approximately 50 percent of the US population has one or more chronic diseases. Chronic disease is the single largest burden to the health care system, accounting for 81% of hospital admissions, 91% of all prescriptions and 76% of physician visits. In a recent CDC National Diabetes Statistics Report, 30.2 million people in the United States are afflicted with chronic diabetes. Most of these people suffer from either obesity, high blood glucose, high blood pressure, high cholesterol, physical inactivity, smoking or a combination of these conditions. The direct and indirect cost of diabetes alone on the health care system amounted to $245 billion, with each patient costing the system $13,700 per year, which is 2.3 times the average of all patients.

Research has shown time and again that patient engagement leads to better care outcomes and reduces cost burden on the healthcare system. However, patient engagement relies on the readiness, and willingness, to take ownership of self-health management. Yet there is a lack of quantitative models to assist in understanding the alignment between the delivery of digital health service and motivation indicators to engage an individual in self-management of chronic diseases.

A system that utilizes behavioral prediction techniques would be helpful in managing patient self-care. Prediction techniques such as linear regression and PCA (Boehmke, B., & Greenwell, B. (2019). Hands-On Machine Learning with R. Chapman and Hall/CRC, ISBN 9781138495685) rely on the linearity of the data of Real in the dimensions that the data reside. These techniques work well when the data distribution exhibits linearity. Unfortunately, the relationship among the behavior constructs (motivation, intention, attitude, and ownership) are not necessarily linear. Many other relationships beyond these behavior constructs also are non-linear.

Conventional computer technology struggles to deal with this complex computational problem. Information-theoretic based techniques such as ID3 (Quinlan, J. R. (1986), Induction of Decision Trees. Machine Learning, 9(1):81-106, 1986), utilizes entropy reduction concept for deriving a decision tree that maximizes information gain in each traversal step of the decision tree. Such a technique does not rely on linearity assumption. However, it is exponential in nature with respect to the number of type enumeration of the multi-dimensional data of finite discrete type. It could be effective when the data distribution lends itself to rapid pruning of impossible cases, or when an association pattern fails (1) a threshold test, and/or (2) non-linear information-theoretic criteria such as the asymptotic convergence of mutual information measure towards Chi-square.

Manifold clustering provides a means to discover data subsets that could be projected to hyperplanes embedded in low dimensions. In other words, a hyperplane is defined by a cluster of a data subset, which is not necessarily linear. In contrast to techniques such as PCA, manifold clustering does not rely on the linearity of the data subset. Manifold clustering techniques such as Spectral Clustering (Kak A., (2018). Low Dimensional Manifold in a High-Dimensional Measurement Space. Data on Manifolds Tutorial, Purdue University), however, suffer from two limitations. First, it is sensitive to the initial seeding for clustering and often requires a 2-phase approach (Luxburg, V. (2007). U. Stat Comput, 17: 395). Second, it could not handle a data set composed of data with mixed data types, for example, data of Real and data of finite discrete type. Even if these limitations could be overcome, manifold clusters are often difficult to interpret without incorporating information-theoretic perspective. An improved method of handling non-linear clustering in embedded low dimension space is therefore desired.

The discussion above is merely provided for general background information and is not intended to be used as an aid in determining the scope of the claimed subject matter.

### BRIEF DESCRIPTION OF THE INVENTION

A method for identifying a manifold cluster using statistically significant association patterns is provided. Customized actions are generated that are specific to the manifold cluster.

Statistically significant association patterns for inducing an initial partition of data for deriving manifolds is used. Manifolds are hyperplanes embedded in low dimensions. The disclosed method has significant advantages over conventional computer technology in that the method is a bootstrap on data clusters that reveal hidden statistical associations from the information-theoretic perspective. This is accomplished without needing to predefine groups. Conventional computer technology merely groups individuals into predetermined groups, often using heuristic rules and assuming simple, linear relationships. This significantly increases the number of customized actions that must be predesigned (e.g one for each group, the number of which can be extremely large). Furthermore, conventional computer technology incorrectly links a given individual to one of the predetermined groups when heuristic rules or assumptions are not a reflection of the real world scenarios and, as such, the customized action is often misaligned with the individual. As a further disadvantage, non-linear prediction techniques often require significantly more extensive resources from traditional computer technology in terms of storage and processing power due to the inherent complexity of the problem and to achieve practical efficiency and accuracy.

The method is particularly useful in enhancing patient compliance with a medical therapy plan. The disclosed technique is applied to a real data set of diabetes patients. An assessment on the effectiveness of the disclosed method is performed to show the effect of bootstrapping based on association patterns.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is flow diagram showing workflow for a method of optimizing patient engagement on self-health management.

### DETAILED DESCRIPTION OF THE INVENTION

This disclosure presents a manifold clustering approach based on the concept of statistically significant association patterns. Conventionally, identifying such hidden sub-populations is a daunting task. This disclosure addresses at least some of the prior art's limitations via the following approach: (1) Data of Real (e.g., continuous real numbers representing a patient's motivation, intention, attitude and ownership) is discretized via an entropy approach that optimizes the trade-off between information loss and the granularity of the discrete representation of the information carried by the data of Real. The discrete representation of the data of Real then enables the discovery of statistically significant association patterns, which are detailed elsewhere in this specification (2) each of the statistically significant association patterns then induces an initial cluster for aggregating data within the proximity that characterizes the hyperplane embedded in the low dimension. This initial cluster then serves as initial seeding for clustering when applying techniques such as spectral clustering, and allows a semantic interpretation from the information-theoretic perspective. (Sy, B. (2019). “Incorporating Association Patterns into Manifold Clustering for Enabling Predictive Analytics,” 2019 International Symposium on Data Science, Las Vegas, Dec. 5-7, 2019).

The significance of the disclosed manifold clustering is the ability to provide a semantic meaning on the clusters based on the concept of association patterns. Conventional computer technologies fail to provide such a predictive benefit. Specifically, each cluster is a collection of data that are “closest” to a statistically significant association pattern in terms of semantic similarity as measured by membership function. By referencing the definition of statistically significant association pattern (Sy, B., & Gupta A. (2004). Information-Statistical Data Mining: Warehouse Integration with Examples of Oracle Basics. eBook ISBN: 978-1-4419-9001-3, DOI: 10.1007/978-1-4419-9001-3, Springer), such a pattern manifests a frequent occurrence as defined by the support measure exceeding a predefined threshold, as well as an inter-relationship among the underlying variables of the pattern that deviates from independence as measured by mutual information from the perspective of information theory. In the use case example of patient engagement, individuals who, based on their answers to the survey question, have statistically significant association patterns are grouped into like clusters. Customized self-monitoring coaching that is specific to a given cluster can thus target specific clusters of patients for maximum effect. Advantageously, the clusters are not predefined but, instead, are dynamically generated as part of the disclosed method itself.

In contrast with the disclosed method, conventional computer technologies generally attempt to address this issue by training an artificial intelligence (AI) or by modeling the system such that the individuals can be fitted into predefined groups. The AI-based solution suffers from AI bias, wherein the result is based on the input data that was used to train the AI. This often results in individuals being incorrectly classed in an inappropriate group. Modeling the system using predefined groups presents a different set of problems. If a small data set is used to construct the model, then the resulting groups are too small in number and lack specificity to properly address the members of the group. If a large data set is used (e.g. “Big Data”) the data is so significant that the increased computational complexity could render the feasibility of a practical solution and increase the costs which, in turn, places the service out of the price range of many consumers. This is true even when the data distribution exhibits linearity. Non-linear solutions, such as K-means, are also available in some situations. However, k-means breaks down in higher dimensions as the k-value would need to be predefined for a multitude of cases. To address these shortcomings, the disclosed method allows one to investigate the effect of dimension reduction on information loss from information-theoretic perspective, as well as from a reconstruction error perspective during the projection of a data point to a hyperplane of a cluster.

In this disclosure the manifold clustering based on association patterns is applied to personalized health coaching. A pilot study on engaging individuals on self-health management using SIPPA Health Informatics Platform was conducted to illustrate the feasibility of affecting behavior change towards a healthy lifestyle (Sy, B., (2018) STTR Phase II: Self-Health Management Informatics Platform: Improving Patient Engagement in Care Delivery. Award Abstract #1831214, NSF). In this pilot study, a validated survey instrument (Sy, B., (2017). SEM Approach for TPB: Application to Digital Health Software and Self-Health Management, 2017 International Symposium on Health Informatics and Biomedical Systems, Las Vegas. Dec. 14-16, 2017) is used to discover the behavior readiness measure of an individual to be engaged in actionable health activities. Behavior readiness measure is a vector of Real characterizing four behavior constructs [motivation, intention, attitude, ownership]. On a daily basis actionable health recommendations, which range from daily advice on healthy diet, setting goals on physical activities, to self-monitoring of vitals such as blood glucose/pressure readings, were sent to an individual.

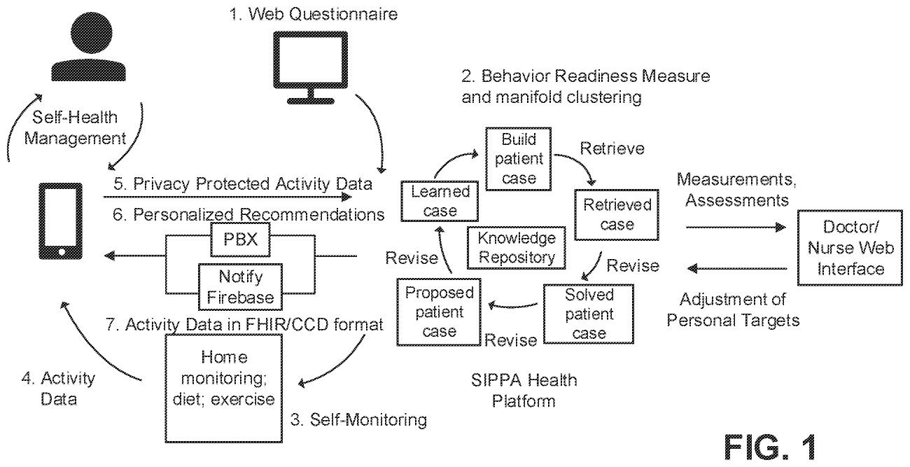

Daily messages were sent via push notifications, in-app service, or a phone call scheduled and routed through a telephone exchange system PBX, to a subject's mobile device. A subject is then asked to provide feedback on each message in terms of “like” (equivalent to useful), “dislike” (not useful), and “dismiss” (neutral). If a daily message is actionable such as self-monitoring of glucose level, the subject is expected to carry out the self-monitoring activities. FIG. 1 illustrates the workflow of an illustrative application of the method.

In the embodiment shown in FIG. 1, behavior readiness is measured (see International Patent Publication WO2019/068086 for details, the content of which is hereby incorporated by reference). Behavior readiness is then used by the manifold cluster process described below to identify subgroups with statistically significant association patterns of behavior. By segmenting into subgroups that exhibit similar behavior readiness patterns, self-care plans specific to a given subgroup are customized and delivered. Advantageously, a reduced number of more accurate and targeted self-care plans can therefore be generated because each self-care plan is specific to a given subgroup. As a further advantage, the self-care plans are customized such that they are targeted to the given subgroup to maximize impact. As discussed elsewhere in this specification, self-monitoring shown in (3) of FIG. 1 may include monitoring physiological parameters using devices such as glucose meters, continuous glucose meters, thermometers, pulse oximetry meters, weight scales, blood pressure meters and the like. Self-monitoring may also include recording exercise sessions and/or food consumption and diet.

As shown in (4) of FIG. 1, the activity data is recorded in a mobile computing device. For example, the physiological parameters are recorded in a mobile computing device.

As shown in (5) of FIG. 1, the activity data is encrypted and sent to the behavior readiness measurement module. The behavior readiness measurement module returns, in step 6, personalized recommendations to the mobile computing device. The patient's compliance with self-monitoring can thus be altered. The personalized recommendations may be sent through a Private Branch Exchange (PBX) messaging system and may be in the form of a text message, an audio voice mail, or other similar communication. In one embodiment, the smart phone is configured to connect back to the system for further medical assistance, such as an embedded video chat system feature to connect a patient with a healthcare provider.

Manifold clustering based on association patterns is comprised of four tasks; first deriving the corresponding discrete data representation of a given set of data of Real. Second, identifying the statistically significant association patterns of the discrete data representation. Third, assigning each data point of real to a cluster based on the evaluation of the membership function of its corresponding discrete data representation against every statistically significant association pattern; fourth, deriving the data clustering on manifold by minimizing reconstruction error.

This disclosure focuses on illustrating a use case of the disclosed method for the predictive analytics based on disclosed manifold clustering to identify non-trivial subgroups of pilot participants who are responsive to the daily messages. The testbed for this preliminary study was a sample collection of data from 53 individuals for the behavior attributes, and among them eight have participated in the pilot for almost three months. The average number of days of participation among the eight is 96.43 days.

A compliance index was derived for measuring the average responsiveness of a subject to the push notifications over a subject participation period. The self-monitoring compliance index is defined as the (average) number of self-monitoring per day divided by the number of self-monitoring per day recommended by a physician according to the clinical guidelines and the diabetes condition of an individual. Similarly, the daily wisdom compliance index is defined as the number of responses to daily wisdom divided by the number of daily wisdom sent over to the subject during the subject's participation period. Daily wisdom consists of healthy tips from a pool of over 100 messages; e.g., “Getting enough sleep is critical to keeping stress under control.” This is in addition to a push notification that could carry an actionable message such as “It's time to self-monitor your glucose level and sync the reading to your personal health record.”

The training data set just mentioned is used to derive the manifold spaces. The task of predictive analytics is to identify the manifolds in the embedded subspace that are induced by statistically significant association patterns and define the clusters of the pilot participants. In other words, the spanning space is a 6-dimensional space composed of behavior constructs [motivation, intention, attitude, ownership], together with self-monitoring compliance index and daily wisdom compliance index.

### Notation, Definition, and Problem Formulation

Let Xⁿ={Xᵢ|Xᵢ∈Rⁿ for i=1 . . . N} be a data set of Real. For example, a given patient may have a data set of [0.76, 0.69, 0.86, 0.81] for motivation, intention, attitude and ownership (N=4) based on that patient's answers to the survey.

Let Yⁿ={Yᵢ|Yᵢ∈Zⁿ for i=1 . . . K≤N} be a data set of finite discrete non-negative Integer.

Let M={Mₖ|Mₖ⊆Xⁿ for k=1 . . . m} be the set of clusters.

Let F:Xⁿ→Yⁿ be a one-on-one bijective mapping function that defines the mapping of the multivariate data set Xⁿ to finite discrete non-negative integers.

Let S(Mₖ)={Pⱼ^{k,o}|Given a k^{th} cluster Mₖ, Pⱼ^{k,o}=(val_{j,1}^{k,o}, . . . , val_{j,o}^{k,o}) is an o^{th} order (2≤o≤n) statistically significant association pattern for j=1 . . . |S(Mₖ)|};

whereas Pⱼ^{k,o}=(val_{j,1}^{k,o}, . . . , val_{j,o}^{k,o}) is a statistically significant association pattern (Sy, B., & Gupta A. (2004). Information-Statistical Data Mining: Warehouse Integration with Examples of Oracle Basics. eBook ISBN: 978-1-4419-9001-3, DOI: 10.1007/978-1-4419-9001-3, Springer)
when Pr(val_{j,1}^{k,o}, . . . , val_{j,o}^{k,o})>threshold, and

MI(val_{j,1}^{k,o}, . . . , val_{j,o}^{k,o})→χ² as defined below:

$$MI ( {val}_{j , 1}^{k , o} , … , {val}_{j , o}^{k , o} ) \to ( \frac{1}{Pr ( {val}_{j , 1}^{k , o} ) Pr ( {val}_{j , 2}^{k , o} ) … Pr ( {val}_{j , o}^{k , o} )} ) {( \frac{{X}^{2}}{2 N} )}^{{( \frac{\hat{E}}{E ′} )}^{O / 2}} where \\ MI ( {val}_{j , 1}^{k , o} , … , {val}_{j , o}^{k , o} ) = \frac{{Log}_{2} ( {val}_{j , 1}^{k , o} , … , {val}_{j , o}^{k , o} )}{Pr ( {val}_{j , 1}^{k , o} ) Pr ( {val}_{j , 2}^{k , o} ) … Pr ( {val}_{j , o}^{k , o} )} \qquad ( 1 )$$

N is the sample size
χ² is the Pearson chi-square defined as (oᵢ−eᵢ)²/eᵢ
Ê is the expected entropy measure
E′ is the maximum possible entropy

Definition 1

The scope coverage of a pattern Pⱼ^{k,o}, represented by SC(Pⱼ^{k,o}), is defined as a subset of Y in which the logical interpretation of every element in the subset is true.

### Example

Let Y={[d1:0 d2:0 d3:0 d4:0], [d1:0 d2:0 d3:0 d4:1], . . . , [d1:1 d2:1 d3:1 d4:1]}, and

P_{q}^{k,o}=[d1:1, d3:0] (for q=1 and o=2) a statistically significant association pattern of the k^{th} cluster.

The scope coverage of P_{q}^{k,o}=[d1:1, d3:0] is denoted by

$$SC ( {P}_{q}^{k , o} ) = \{ [ d 1 \text{:} 1 , d 2 \text{:} 0 , d 3 \text{:} 0 d 4 \text{:} 0 ] , [ d 1 \text{:} 1 , d 2 \text{:} 0 , d 3 \text{:} 0 , d 4 \text{:} 1 ] , [ d 1 \text{:} 1 , d 2 \text{:} 1 , d 3 \text{:} 0 d 4 \text{:} 0 ] , . [ d 1 \text{:} 1 , d 2 \text{:} 1 , d 3 \text{:} 0 d 4 \text{:} 1 ] \}$$

Definition 2

The membership function ƒ(P_{q}^{k,o}, Pⱼ^{k′,o′})→[0,1] is defined by the geometric mean measure below:

$$f ( {P}_{q}^{k , o} , {P}_{r}^{{k}^{′} , {o}^{′}} ) = \sqrt{\begin{matrix}\frac{\vert SC ( {P}_{q}^{k , o} ) ⋂ SC ( {P}_{j}^{k ′ , o ′} ) \vert}{\vert SC ( {P}_{q}^{k , o} ) \vert} \times \\ \frac{\vert SC ( {P}_{q}^{k , o} ) ⋂ SC ( {P}_{j}^{k ′ , o ′} ) \vert}{\vert SC ( {P}_{j}^{k , o} ) \vert}\end{matrix}}$$

Pⱼ^{k′,o′} is a member of the k^{th} manifold induced by P_{q}^{k,o} when k=ArgMax_{q,k}ƒ(P_{q}^{k,o},Pⱼ^{k′,o′}).

### Example

Assume P₁^{1,2}=[d1:1, d3:0] P₂^{2,3}=[d1:0, d3:1,d4:1] P₃^{k′,3}=[d1:1,d3:0,d4:1]

The following terms are derived based on the definitions:

$$SC ( {P}_{1}^{1 , 2} ) = \{ [ d 1 \text{:} 1 , d 2 \text{:} 0 , d 3 \text{:} 0 d 4 \text{:} 0 ] , [ d 1 \text{:} 1 , d 2 \text{:} 0 , d 3 \text{:} 0 , d 4 \text{:} 1 ] , [ d 1 \text{:} 1 , d 2 \text{:} 1 , d 3 \text{:} 0 d 4 \text{:} 0 ] , [ d 1 \text{:} 1 , d 2 \text{:} 1 , d 3 \text{:} 0 d 4 \text{:} 1 ] \}$$

$$SC ( {P}_{2}^{2 , 3} ) = \{ [ d 1 \text{:} 0 , d 2 \text{:} 0 , d 3 \text{:} 1 d 4 \text{:} 1 ] , [ d 1 \text{:} 0 , d 2 \text{:} 1 , d 3 \text{:} 1 , d 4 \text{:} 1 ] \} . \\ SC ( {P}_{3}^{k ′ , 3} ) = \{ [ d 1 \text{:} 1 , d 2 \text{:} 0 , d 3 \text{:} 0 d 4 \text{:} 1 ] , [ d 1 \text{:} 1 , d 2 \text{:} 1 , d 3 \text{:} 0 , d 4 \text{:} 1 ] \}$$

$$f ( {P}_{1}^{1 , 2} , {P}_{3}^{{k}^{′} , 3} ) = \sqrt{\begin{matrix}\frac{\vert SC ( {P}_{1}^{1 , 2} ) ⋂ SC ( {P}_{3}^{k ′ , 3} ) \vert}{\vert SC ( {P}_{1}^{1 , 2} ) \vert} \times \\ \frac{\vert SC ( {P}_{1}^{1 , 2} ) ⋂ SC ( {P}_{3}^{k ′ , 3} ) \vert}{\vert SC ( {P}_{3}^{k ′ , 3} ) \vert}\end{matrix}} = \sqrt{( \frac{2}{4} ) ( \frac{2}{2} )}$$

$$f ( {P}_{2}^{2 , 3} , {P}_{3}^{{k}^{′} , 3} ) = \sqrt{\begin{matrix}\frac{\vert SC ( {P}_{3}^{2 , 3} ) ⋂ SC ( {P}_{3}^{k ′ , 3} ) \vert}{\vert SC ( {P}_{2}^{2 , 3} ) \vert} \times \\ \frac{\vert C ( {P}_{2}^{2 , 3} ) ⋂ SC ( {P}_{3}^{k ′ , 3} ) \vert}{\vert {P}_{3}^{k ′ , 3} \vert}\end{matrix}} = \sqrt{( \frac{0}{2} ) ( \frac{0}{2} )}$$

P₃^{k′,3} is member of the manifold induced by P₁^{1,2} because

$${ArgMax}_{k , q} f ( {P}_{q}^{k , o} , {P}_{3}^{k ′ , 3} ) = 1$$

Algorithm for deriving discrete data representation of data of Real

Consider a discrete variable Y of N possible states, the entropy of a system defined by Y−H_{N}(P₁ . . . P_{N}) (where Pⱼ=Pr(Y=yᵢ) for j=1 . . . N) is

$${H}_{N} ( {P}_{1} … {P}_{N} ) = {\sum }_{i}^{ } - Pr ( Y = {y}_{i} ) {Log}_{2} Pr ( Y = {y}_{i} ) = {\sum }_{i} - {P}_{i} {Log}_{2} {P}_{i}$$

It can be shown that the following equality holds (Sy, B., & Gupta A. (2004). Information-Statistical Data Mining: Warehouse Integration with Examples of Oracle Basics. eBook ISBN: 978-1-4419-9001-3, DOI: 10.1007/978-1-4419-9001-3, Springer):

$${H}_{N} ( {P}_{1} … {P}_{N} ) = {H}_{N - 1} ( {P}_{1} + {P}_{2} , {P}_{3} … {P}_{N} ) + ( {P}_{1} + {P}_{2} ) {H}_{2} ( \frac{{P}_{1}}{{P}_{1} + {P}_{2}} , \frac{{P}_{2}}{{P}_{1} + {P}_{2}} )$$

In the quantization process, combining two terms will reduce the number of terms by one, and at the same time results in an information loss amounting to the second term on the right-hand side of the above equation.

The quantization of a data set of Real disclosed in this disclosure utilizes the entropy equation just shown that incrementally combines terms until it reaches the reflection point where there is a change of direction on the rate of change of information loss. The details of the algorithm is shown below:

Let Xⁿ={Xᵢⁿ|Xᵢⁿ∈Rⁿ for i=1 . . . N} be a data set of Real. For each dimension j=1 . . . n of Xⁿ, perform the following steps for the data of the j^{th} dimension:

Step 1: Order Xᵢ^{j} in an ascending order. Create a bucket/bin for each term in X^{j}. Treat each bucket/bin as a state of a discrete variable of Y and associate a value for a bucket/bin equal to the mean of the term(s) in the bucket/bin. In other words, Y is a discrete variable of N states. If the values of Xᵢ^{j} are all different, the distribution of Y is then even and the probability of Y for every term is equal to I/N.

Step 2: Initialize an iteration count C=1. Derive the entropy H_{N}(P₁ . . . P_{N}) and record it as H_{N}^{C}.

Step 3: Increment the iteration count by 1; i.e., C=C+1. Identify two adjacent buckets/bins, say, the j^{th} and (j+1)^{th} in the ordered list where the difference between the mean of the terms in the jth bucket/bin and that in the (j+1)^{th} is the smallest. Combine the two adjacent buckets/bins into one. Update the mean of the data in the combined bucket/bin, and update the probability distribution of Y. Re-derive the entropy H_{N-1}^{C+1}. Record the information loss I^{C+1} due to combining the two terms; i.e.,

$${I}^{C + 1} = ( {P}_{j} + {P}_{j + 1} ) {H}_{2} ( \frac{{P}_{j}}{{P}_{j} + {P}_{j + 1}} , \frac{{P}_{j + 1}}{{P}_{j} + {P}_{j + 1}} )$$

Step 4: Repeat step 3 until a terminal criteria is reached (e.g. the direction on the rate of change of I^{C+j} is changed or an inflection point). When this occurs at the kth iteration, the following result is obtained:

$${X}^{n} = \{ {X}_{i}^{n} \vert {X}_{i}^{n} ∈ {R}^{n} for i = 1 … N \}$$

$${Y}^{n} = \{ {Y}_{i} \vert {Y}_{i} ∈ {Z}^{n} for i = 1 … K \leq N \} be a data set .$$

Let F: Xⁿ→Yⁿ be an one-on-one bijective mapping function that defines the discretization of the multivariate data set X. Below shows an example:

$${X}^{n} = \{ [ 0.76 , 0.69 , 0.86 , 0.81 ] , [ 0.87 , 0.49 , 0.72 , 0.98 ] , [ 0.11 , 0.67 , 0.57 , 0.75 ] , [ 0.39 , 0.71 , 0.63 , 0.82 ] \}$$

$$F ( [ 0.76 , 0.69 , 0.86 , 0.81 ] ) = [ 1 , 1 , 2 , 1 ]$$

$$F ( [ 0.87 , 0.49 , 0.72 , 0.98 ] ) = [ 2 , 0 , 1 , 2 ]$$

$$F ( [ 0.11 , 0.67 , 0.57 , 0.75 ] ) = [ 0 , 1 , 0 , 0 ]$$

$$F ( [ 0.39 , 0.71 , 0.63 , 0.82 ] ) = [ 1 , 2 , 0 , 1 ]$$

$${Y}^{n} = \{ [ 1 , 1 , 2 , 1 ] , [ 2 , 0 , 1 , 2 ] , [ 0 , 1 , 0 , 0 ] , [ 1 , 2 , 0 , 1 ] \}$$

Algorithm for deriving data clustering on manifold

Given Xⁿ, Yⁿ, and F, and a predefined error threshold 6, the algorithm for the disclosed manifold clustering based on statistical significant association patterns is shown below;

Step 1: Based on Yⁿ={Yᵢ|Yᵢ∈Zⁿ for i=1 . . . K≤N}, derive the set of statistically significant association patterns; i.e., S(Mₖ)={Pⱼ^{k,o}|Given a k^{th} cluster Mₖ, Pⱼ^{k,o}=(val_{j,1}^{k,o}, . . . , val_{j,o}^{k,o}) is an o^{th} order (2≤o≤n) statistically significant association pattern for j=1 . . . |S(Mₖ)|};

Pⱼ^{k,o}=(val_{j,1}^{k,o}, . . . , val_{j,o}^{k,o}) is a statistically significant association pattern when Pr(val_{j,1}^{k,o}, . . . , val_{j,o}^{k,o})>threshold, and

MI(val_{j,1}^{k,o}, . . . , val_{j,o}^{k,o})→adjusted χ² as defined below:

$$MI ( {val}_{j , 1}^{k , o} , … , {val}_{j , o}^{k , o} ) \to ( \frac{1}{Pr ( {val}_{j , 1}^{k , o} ) Pr ( {val}_{j , 2}^{k , o} ) … Pr ( {val}_{j , o}^{k , o} )} ) {( \frac{{X}^{2}}{2 N} )}^{{( \frac{\hat{E}}{E ′} )}^{O / 2}} where$$

$$MI ( {val}_{j , 1}^{k , o} , … , {val}_{j , o}^{k , o} ) = \frac{{Log}_{2} ( {val}_{j , 1}^{k , o} , … , {val}_{j , o}^{k , o} )}{Pr ( {val}_{j , 1}^{k , o} ) Pr ( {val}_{j , 2}^{k , o} ) … Pr ( {val}_{j , o}^{k , o} )}$$

N is the sample size
χ² is the Pearson chi-square defined as (oᵢ−eᵢ)²/eᵢ
Ê is the expected entropy measure
E′ is the maximum possible entropy

Step 2: Define |M| disjoint clusters such that each cluster has one and only one statistically significant association pattern. Let W be the set of cluster reference holding the data points in X; i.e., W={X^{n,j}|X=∪ⱼX_{n,j} for n=1 . . . |S(Mₖ)|}.

Step 3: Partition X by assigning each data point Xᵢ to the cluster X^{n,k} if ArgMax_{q,k}ƒ(F(Xᵢ),P_{q}^{k,o})=k; where P_{q}^{k,o} is a pattern that defines the cluster Mₖ, thus X^{n,k}. ƒ is the membership function defined previously. If ƒ(F(Xᵢ), P_{q}^{k,o}) is zero in all cases, Xᵢ is assigned to a non-semantic cluster NS. In this manner, the number of clusters is determined.

Step 4: Let S={Sⱼ|j=1 . . . |S(Mₖ)|} be the set of subspaces corresponding to the clusters defined in step 2. Repeat the following for each j where the corresponding cluster has more than one element:

Let D^{n,j}={dₖ^{n,j}|k=1 . . . |X^{n,j}|} be the data set of the cluster X^{n,j}. The subspace Sⱼ corresponding to X^{n,j} is then derived based on the following:

Step 4.1: Derive the mean vector and variance matrix of D^{n,j} for each j=1 . . . |S(Mₖ)| i.e.,

$${M}^{n , j} = ( \frac{1}{\vert {D}^{n , j} \vert} ) {\sum }_{k = 0}^{\vert {D}_{k}^{n , j} \vert} ( {d}_{k}^{n , j} - {m}^{n , j} ) {( {d}_{k}^{n , j} - {m}^{n , j} )}^{T} where$$

$${m}^{n , j} = ( ( 1 / \vert {D}^{n , j} \vert ) {\sum }_{k = 0}^{\vert {D}_{k}^{n , j} \vert} ( {d}_{k}^{n , j} )$$

Step 4.2: Conduct eigendecomposition on M^{n,j} to obtain the eigenvector matrix Q^{n,j} and the eigenvalue matrix ∧^{n,j} such that M^{n,j}=(Q^{n,j})∧^{n,j}(Q^{n,j})⁻¹.

Step 4.3: Let P be the number of non-zero eigenvalues obtained in step 4.2.

Step 4.4: Use the P′ (leading) eigenvectors in Q^{n,j} to define the local coordinate frame for the subspace Sⱼ, and rewrite Q^{n,j}=[W^{P′,j}W^{n-p′,j}].

Step 4.5: The projection error of mapping a data point dₖ^{n,j} to the subspace Sⱼ defined by the local coordinate frame is then equal to e=(W^{n-p′,j})^{T}(dₖ^{n,j}−m^{n,j}). Or the square-magnitude projection error of dₖ^{n,j} to the subspace Sⱼ is then equal to Err(dₖ^{n,j},Sⱼ)=(dₙ^{n,j}−m^{n,j})^{T}(W^{n-p,j}) (W^{n-p,j})^{T}(dₖ^{n,j}−m^{n,j}). Calculate the total error: Σⱼ Σₖ Err(dₖ^{n,j},Sⱼ).

Step 4.6: Repeat step 4.4 and step 4.5 with a new P that is one less; i.e., P−1. Record the total error.

Step 4.7: Compute the total reconstruction error ratio of two successive rounds in step 4.6; i.e., (total reconstruction error using P−q−1 leading eigenvector)/(total reconstruction error using P−q leading eigenvector) where q=0 . . . P−2.

Step 4.8: Finalize the local coordinate frame for the subspace Sⱼ with a dimension P−q when the error ratio in step 4.7 is the largest for the given q.

Step 5: An error calculation is utilized to determine if the same (or an acceptably similar) degree of error can be obtained with fewer clusters. If fewer clusters can be utilized, while maintaining an acceptable error, then computer processing expenses can be reduced. Merge two or more clusters that do not involve NS. If there are clusters with only one data point, these clusters will take the priority; then repeat step 4. Retain the solution with a lower total error.

Step 6: Repeat step 5 until the total error is below the predefined error threshold S, or the algorithm reaches the maximum number of iterations allowed.

One noteworthy observation on the step 5 of the algorithm above is that the merged cluster will be characterized by not one, but multiple statistically significant association patterns, and the meaning of a data point in terms of its closeness to the semantic interpretation of some association pattern in a merged cluster, in terms of scope coverage and membership function, is still preserved.

Experimental Results and Analysis

Behavior readiness measure is a 1×4 vector of Real composed of behavior constructs [motivation, intention, attitude, ownership]. There are 53 such vectors, and each vector is discretized to become a vector of finite discrete values using the disclosed algorithm. The vectors of discrete values become the data set for discovering statistically significant association patterns based on equation 1. Using a support measure threshold 0.2, 25 statistically significant association patterns are found and listed in Table 1.

TABLE 1
Statistically Significant Association Patterns with Self-Monitoring
and Daily Wisdom that induces manifold clusters. Percentage of patterns
identified as statistically significant: 25/1296 = 1.92%

| | Motiva- | Inten- | | Owner- | Self- | Daily |
|---|---|---|---|---|---|---|
| | tion | tion | Attitude | ship | Monitoring | Wisdom |
| Pattern 1 | | | 2 | 1 | | |
| Pattern 2 | | 1 | | I | | |
| Pattern 3 | | 2 | 0 | | | |
| Pattern 4 | | 1 | 1 | | | |
| Pattern 5 | 2 | | | 1 | | |
| Pattern 6 | 2 | | 1 | | | |
| Pattern 7 | 2 | | 1 | 1 | | |
| Pattern 8 | 2 | 1 | | | | |
| Pattern 9 | | | | I | | 1 |
| Pattern 10 | | | 2 | | | 1 |
| Pattern 11 | | | 1 | | | 2 |
| Pattern 12 | | 1 | | | | 1 |
| Pattern 13 | 1 | | | | | 1 |
| Pattern 14 | | | | 1 | 1 | |
| Pattern 15 | | | | 1 | 0 | |
| Pattern 16 | | | 0 | | 1 | |
| Pattern 17 | | | 1 | 1 | 1 | |
| Pattern 18 | | 1 | | | 1 | |
| Pattern 19 | | 2 | | | 1 | |
| Pattern 20 | | 1 | | 1 | 1 | |
| Pattern 21 | 2 | | | | 1 | |
| Pattern 22 | 0 | | | | 1 | |
| Pattern 23 | 2 | | | 1 | 1 | |
| Pattern 24 | | | | | 1 | 1 |
| Pattern 25 | | | | 1 | 1 | 1 |

*D-motivation: {0, 1, 2}, D-intention = {0, 1, 2, 3}, D-attitude = {0, 1, 2}, D-ownership {0, 1, 2}, D-daily-wisdom-engagement: {0, 1, 2, 3}, D-self-monitoring: {0, 1, 2}

By way of illustration, and not limitation, pattern 1 in Table 1 shows an attitude score of 2 and an ownership score of 1 is a significant association pattern. A customized message may be sent to patients in this specific manifold cluster that is customized to enhance their compliance with self-health management through the PBX messaging system. This customized message is designed to target those individuals with a high attitude score and a moderate ownership score. This same customized message may not be as effective on patients in other manifold clusters and, as such, this particular customized message is not necessarily sent to the patients in other clusters.

One would expect 25 manifold clusters when there are 25 statistically significant association patterns. But in reality there could be fewer non-empty manifold clusters. For example, pattern 25 [ownership:1 self-monitoring:1 daily-wisdom:1] is a special case under the scope covered by pattern 9 [ownership:1 daily-wisdom:1]. And there could be clusters with only one data point when the data set is sparse, which is our case. As a result, only six manifold clusters are non-empty, and four of them contain only one data point as shown in Tables 2 to 4.

TABLE 2
Manifolds with more than one data point for dimension reduction

| | # Eigenvectors | # Discarded | Reconstr. Error |
|---|---|---|---|
| Cluster 5 | 6 | 4 | 0.0241 |
| Total | 6 | 4 | 0.0241 |

TABLE 3
Manifolds (after merging clusters of only
one data point with others in step 5)

| | # Eigenvectors | # Discarded | Reconstr. Error |
|---|---|---|---|
| Cluster 2, 3 | 6 | 5 | 0.0807 |
| Cluster 1, 4, 6 | 6 | 4 | 0.0381 |
| Cluster 5 | 6 | 4 | 0.0241 |
| Total | 18 | 13 | 0.1429 |

TABLE 4
Manifolds (after merging clusters in step 6)

| | # Eigenvectors | # Discarded | Reconstr. Error |
|---|---|---|---|
| Cluster 1, 2, 4, 5 | 6 | 2 | 0.0032 |
| Cluster 3, 6 | 6 | 4 | 0.0241 |
| Total | 12 | 4 | 0.0273 |

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a non-transient computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language, mobile application development such as ANDROID® programming language, front end programming language such as Angular or React Native, or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for identifying a manifold cluster with statistically significant association patterns, the method comprising steps of:
a) receiving a data set of real, continuous numbers of n-dimensions for each individual of a plurality of individuals;
b) converting the real, continuous numbers to corresponding discrete data representations;
b.1) ordering, in numeric order, the continuous numbers, thereby producing an ordered list;
b.2) creating a bucket/bin for each term in the ordered list;
b.3) identifying two adjacent buckets/bins, j^{th} and (j+1)^{th} in the ordered list where the difference between a mean of the terms in the j^{th} bucket/bin and that in the (j+1)^{th} is the smallest;
b.4) combining the two adjacent buckets/bins into one combined bucket/bin and calculating a mean of j^{th} and (j+1)^{th} in the combined bucket/bin, thereby producing a combined, ordered list of terms;
b.5) calculating information loss due to the combining of the two adjacent buckets/bins;
c) repeating steps b.3) to b.5) until a terminal criteria;
d) identifying the statistically significant association patterns of the discrete data representation, thereby producing identified statistically significant association patterns;
e) defining disjoint clusters such that each disjoint cluster has one and only one statistically significant association pattern;
f) assigning each real, continuous number to a disjoint cluster based on evaluation of a membership function of its corresponding discrete data representation against the identified statistically significant association patterns, thereby producing assigned disjoint clusters, wherein the membership function is:
$$f ( {P}_{q}^{k , o} , {P}_{j}^{k ′ , o ′} ) \to [ 0 , 1 ]$$
wherein P_{q}^{k,o} is an association pattern and Pⱼ^{k′,o′} is the j^{th} member of a k^{th} manifold induced by P_{q}^{k,o} when k=ArgMax_{q,k}ƒ(P_{q}^{k,o},Pⱼ^{k′,o′});
g) for each cluster with more than one discrete data representation, defining a subspace for an assigned disjoin cluster by:
g.1) obtaining a number (P) of non-zero eigenvalues in an eigenvector matrix that is obtained from an eigendecomposition of a covariance matrix of each cluster;
g.2) calculating an error resulting from reconstructing the covariance matrix from a low dimension of an embedded space obtained from projecting the continuous data representations onto a space of the respective cluster;
g.3) repeating steps g.1) using P−q leading eigenvectors (where q=0, . . . , P−2) at q iteration and g.2) until the error is minimized, thereby producing a manifold cluster;
h) delivering, by a PBX messaging system, a message to a target individual on a mobile computing device wherein the message is customized based on the manifold cluster that corresponds to the target individual.

2. The method as recited in claim 1, wherein after step g) the method further comprising
i) merging at least two of the clusters;
ii) repeating step g;
iii) comparing the error that was calculated prior to the merging to the error that was calculated after the merging;
iv) repeating steps i) to iii) until the error that was calculated after the merging is within a predefined error threshold 6, or a maximum number of iterations achieved.

3. The method as recited in claim 1, wherein the mobile computing device is a smart phone.

4. The method as recited in claim 3, wherein the message is routed to the smart phone using a Private Branch Exchange (PBX) system.

5. The method as recited in claim 3, wherein the message is a text message.

6. The method as recited in claim 3, wherein the message is an audio voice mail.

7. The method as recited in claim 3, wherein the smart phone is configured to provide video chat with a healthcare provider.

8. The method as recited in claim 3, wherein the smart phone is associated with a specific individual.

9. The method as recited in claim 1, wherein the message is displayed to all individuals in the plurality of individuals that share the manifold cluster with the target individual.

10. The method as recited in claim 1, wherein the data set of real, continuous numbers consists of a motivation score, an intention score, an attitude score and an ownership score.

11. The method as recited in claim 1, further comprising minimizing reconstruction error by:
i. Deriving a mean vector and co-variance matrix A^{n,j} of a data set D^{n,j} of a cluster;
ii. Conducting an eigendecomposition on the A^{n,j} to obtain an eigenvector matrix and an eigenvalue matrix;
iii. Sorting the eigenvalues and re-arrange values in the eigenvalue matrix and corresponding eigenvectors in the eigenvector matrix obtained from ii);
iv. Choosing P′ (<=n) non-zero eigenvalues and splitting the eigenvector matrix into a matrix of leading P′ eigenvectors W^{P′}, and another matrix W^{(n-P′)} consisting of residual (n−P′) eigenvectors.
v. Defining a local coordinate frame for a subspace (Sⱼ) using the leading eigenvector (P′) in the eigenvector matrix W^{P′};
vi. Calculating the square-magnitude projection error of mapping every data point (dₖ^{n,j}) in D^{n,j} to the subspace (Sⱼ) and a total projection error;
vii. Repeating (iv) and (v) with P′=P′−1;
viii. Computing a total reconstruction error ratio of two successive rounds in vi); wherein the minimizing reconstruction error is performed after g) and before h).
